(19) United States
(12) Patent Application Publication
(10) Pub. No.: US 2026/0318834 A1
(43) Pub. Date: Oct. 15, 2026

(54) Digital Pathology-Based Biomarker Detection Using Fluorescent Image Analysis of Chromogenic IHC Stains

(71) Applicant: OmicInsight Corporation, Santa Clara, CA (US)

(72) Inventors: Jekwan Ryu, Cupertino, CA (US); Hojin Lee, Santa Clara, CA (US); Ahreum Oh, San Jose, CA (US)

(21) Appl. No.: 19/646,047

(22) Filed: Apr. 13, 2026

Related U.S. Application Data
(60) Provisional application No. 63/788,058, filed on Apr. 13, 2025.

(51) Int. Cl. G01N 21/64; G01N 1/30; G01N 33/569; G06T 5/50; G06T 5/73

(52) U.S. Cl. CPC G01N 21/6458; G01N 1/30; G01N 33/569; G06T 5/50; G06T 5/73; G01N 2001/302; G01N 2474/20; G06T 2207/10056; G06T 2207/10064; G06T 2207/20224

(57) ABSTRACT

An imaging method provides a high-resolution target spatial map using SAO reconstruction. In one embodiment, the target are proteins in a sample that is labeled according to chromogenic IHC staining. In one embodiment, the proteins are antigens expressed on cells. Optical signals measured by structured excitation of the target may incorporate both signals from nuclei of the cells in addition to signals from proteins. The system and method described herein deconvolves the contribution from cell nuclei from the protein channels, resulting in higher-quality imaging of the protein when reconstructed by SAO. This process can decouple the nuclei signal overlap in the protein channel.

### CROSS-REFERENCE TO RELATED APPLICATIONS

This application claims the benefit of U.S. Provisional Patent Application No. 63/788,058 filed on Apr. 13, 2025, which is incorporated by reference herein in its entirety.

### BACKGROUND OF THE DISCLOSURE

### 1. Field

The present invention relates generally to the field of optical microscopy imaging which uses structured or selective illumination or excitation and, more specifically, to a method of generating protein maps using measured signals from structured illumination patterns.

### 2. Description of the Related Art

Synthetic Aperture Optics (SAO) imaging refers to an optical imaging method in which a series of patterned or structured light patterns are used to illuminate the imaging target in order to achieve resolution beyond what is set by physical constraints of the imaging apparatus such as the lens and the camera. In SAO, an imaging target is selectively excited in order to detect the spatial frequency information of the target. Since there is a one-to-one relationship between the frequency (or Fourier) domain and the object (or target) domain, SAO can reconstruct the original imaging target by obtaining its spatial frequency information.

Conventional clinical biomarker image analysis is performed by imaging a chromogenic immunohistochemistry (IHC) slide in brightfield microscopy. IHC slides may include two stains: DAB (3,3′-diaminobenzidine) for proteins and hematoxylin for nuclei. However, the resulting image is limited in terms of spatial resolution and dynamic range for quantitative analysis at single cell level.

### SUMMARY OF THE DISCLOSURE

Embodiments of the present disclosure include a method for synthetic aperture optics (SAO) that provides high-resolution target spatial maps of, for example, protein. In one embodiment, a method comprises generating a plurality of raw protein images by measuring optical signals from a target illuminated with a first plurality of structured illumination patterns, each raw protein image including at least one raw protein intensity value obtained from measurements of proteins in the target responsive to being illuminated with a corresponding structured illumination pattern. The method further comprises generating a plurality of raw nuclei images by measuring optical signals from the target illuminated with a second plurality of structured illumination patterns, each raw nuclei image including at least one raw nuclei intensity value obtained from measurements of nuclei in the target responsive to being illuminated with a corresponding structured illumination pattern. The method further comprises obtaining a nuclei S image by processing the plurality of nuclei raw images. The method further comprises obtaining a plurality of deconvolved images by computing a difference between each of the protein raw images and the nuclei S image. The method further comprises generating a reconstructed image by processing the plurality of deconvolved images, wherein the reconstructed image includes a set of reconstructed intensity values, wherein the reconstructed image has a higher resolution than the plurality of deconvolved images. The method further comprises generating a protein map from at least the reconstructed image of the target, and further displaying information obtained from the protein map on a client device.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The teachings of the embodiments of the present disclosure can be readily understood by considering the following detailed description in conjunction with the accompanying drawings.

FIG. 1A illustrates a conventional SAO method.

FIG. 1B illustrates a conventional SAO system.

FIG. 1C illustrates an example of a selective excitation pattern in the spatial domain and the frequency domain.

FIGS. 2A, 2B, and 2C illustrate different types of individual sequencing microparticles that can be used for DNA sequencing.

FIGS. 3A and 3B illustrate some examples of the distribution of DNA microparticles.

FIG. 4 illustrates an SAO method, according to one embodiment.

FIG. 5A illustrates the k-space sampling points (selective excitation patterns) used in SAO, according to one embodiment.

FIG. 5B illustrates the selection of the k-space sampling interval used in SAO, according to one embodiment.

FIG. 5C illustrates using selective excitation patterns corresponding to k-space sampling points within a circular region, according to one embodiment.

FIG. 5D illustrates reducing the number of k-space sampling points by sparse k-space sampling, according to one embodiment.

FIG. 6A illustrates how aliasing occurs in SAO by use of a pixel field of view (PFOV) smaller than the detected area, according to one embodiment.

FIG. 6B illustrates how the actual signal at a pixel of an imaging system may be determined by unfolding the measured signal at the pixel to remove aliasing, according to one embodiment.

FIG. 6C illustrates a method of unfolding the measured signal at the pixel to remove aliasing, according to one embodiment.

FIG. 7A illustrates a structured illumination apparatus for selectively exciting the microparticles, according to one embodiment.

FIG. 7B illustrates the arrangement of the illumination pattern generation modules in a half-ring structure, according to one embodiment.

FIG. 7C illustrates the internal structure of an illumination pattern generation module, according to one embodiment.

FIG. 7D illustrates the internal structure of an illumination pattern generation module, according to another embodiment.

FIG. 8 illustrates labeling method of a target via chromogenic immunohistochemistry (IHC) staining, according to one embodiment.

FIG. 9 illustrates an acquisition system for acquiring raw images from a target, according to one embodiment.

FIG. 10 illustrates a target spatial map imaging method using SAO, according to another embodiment.

FIG. 11 illustrates a target spatial map imaging method using SAO and deconvolving signals from nuclei, according to another embodiment.

FIG. 12 illustrates examples of imaging methods that were performed human breast cancer tissue samples, according to one embodiment.

### DETAILED DESCRIPTION OF EMBODIMENTS

The Figures (FIG.) and the following description relate to preferred embodiments of the present invention by way of illustration only. It should be noted that from the following discussion, alternative embodiments of the structures and methods disclosed herein will be readily recognized as viable alternatives that may be employed without departing from the principles of the claimed invention.

Reference will now be made in detail to several embodiments of the present invention(s), examples of which are illustrated in the accompanying figures. It is noted that wherever practicable similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

The imaging method according to various embodiments of the present disclosure provides a high-resolution target spatial map using SAO reconstruction. In one embodiment, the target are proteins in a sample that is labeled according to chromogenic IHC staining. In one embodiment, the proteins are antigens expressed on cells. Optical signals measured by structured excitation of the target may incorporate both signals from nuclei of the cells in addition to signals from proteins. The system and method described herein deconvolves the contribution from cell nuclei from the protein channels, resulting in higher-quality imaging of the protein when reconstructed by SAO. This process can decouple the nuclei signal overlap in the protein channel. In addition, the system and method described herein obtains measured signals for background channels that are indicative of autofluorescence signals occurring from endogenous processes of the cells. The signals from the background channels are used to weigh the intensity values in the reconstructed image to account for noise due to autofluorescence contribution in the target spatial map.

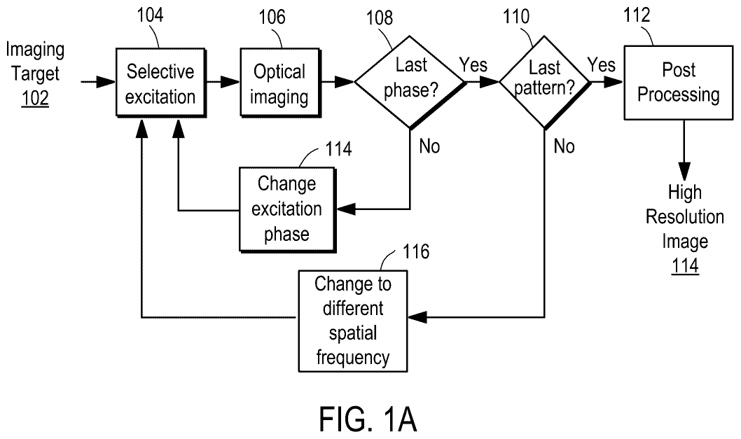

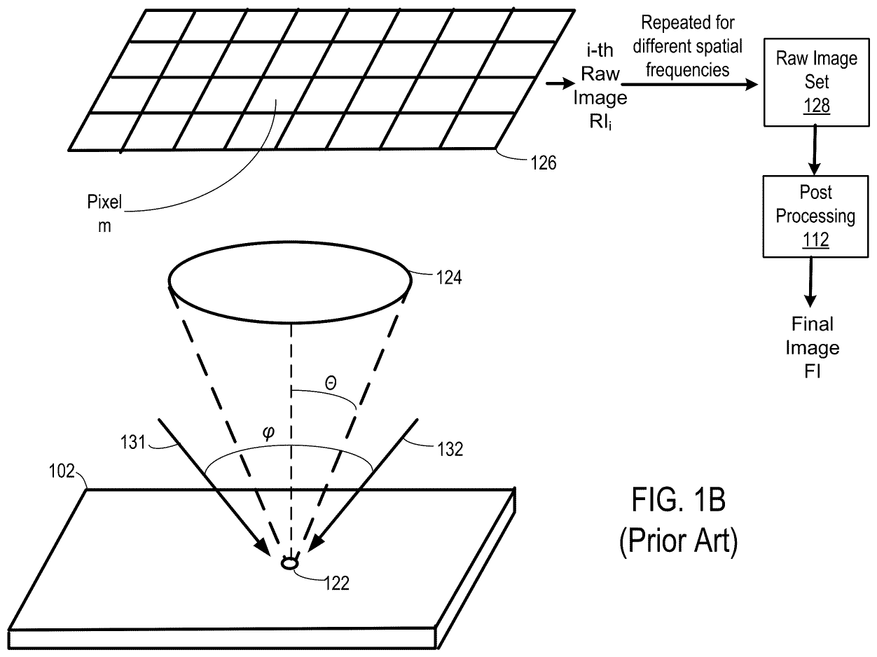

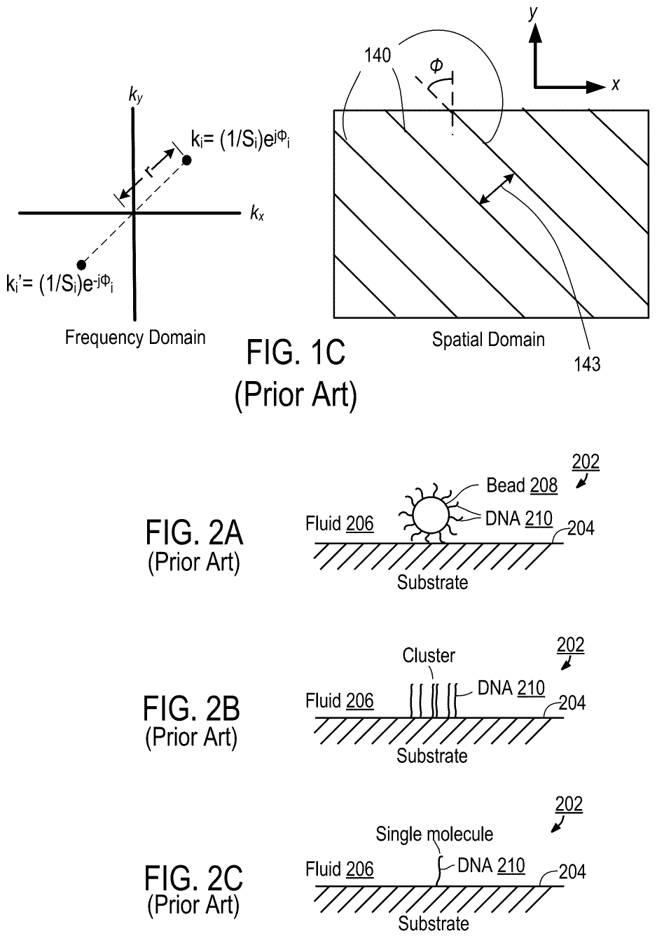

FIG. 1A illustrates a conventional SAO method, and FIG. 1B illustrates a conventional SAO system. Referring to FIGS. 1A and 1B together, in conventional SAO, selective excitation (or illumination) 104 is applied to an imaging target 102, and the light scattered or fluoresced from the imaging target 102 is captured by optical imaging 106. The imaging target 102 can be composed of micro-particles in a randomly or regularly distributed pattern. Selective excitation (or illumination) 104 may be applied to the imaging target 102 by an illumination apparatus (not shown in FIGS. 1A and 1B) that is configured to cause interference 122 of two or more light beams 131, 132 on the imaging target 102. The excitation is selective or patterned, unlike uniform illumination used in conventional optical imaging techniques. For example, two beams 131, 132 may overlay or interfere on an imaging-target plane 102 to produce a two-dimensional (2D) sinusoidal excitation pattern.

FIG. 1C illustrates an example of a selective excitation pattern in the spatial domain and the frequency domain. Referring to FIGS. 1B and 1C, the exemplary selective excitation pattern 140 in the spatial domain is generated by interference of two beams 131, 132 on the imaging-target plane 102, resulting in a 2D sinusoidal excitation pattern. The angle (φ) between the two beams 131, 132 determines the pitch 143 of the pattern, which represents the spacing or periodicity of 2D sinusoidal fringe pattern 140. More specifically, the pitch 143 is substantially inversely proportional to sin(φ). The orientation φ of the pattern represents the amount of angular rotation of the 2D sinusoidal fringes 140 compared to its reference pattern, which in this example of FIG. 1C is shown as a 2D sinusoid comprised of vertical lines, although a different reference pattern such as a 2D sinusoid comprised of horizontal lines can also be used as the reference pattern. In mathematical terms, the orientation φ can be described as follows: if u is the normal vector of the plane formed by the two beams 131, 132 and if the projected vector of u on the imaging plane 102 is called v, then the orientation φ of the sinusoidal pattern 140 is the angular orientation of the vector v with respect to the frame of reference. The “phase” of the pattern is the periodic position of the 2D sinusoid with respect to the frame of reference. The range of the phases of the 2D sinusoid excitation pattern will be a value between 0 and 2π. The different phases can be obtained by changing optical path length of one beam.

As shown in FIG. 1C, the 2D sinusoid excitation pattern in the spatial domain can be shown as a conjugate pair kᵢ, kᵢ′ in the corresponding frequency domain (k-space). Each conjugate pair in the k-space corresponds to the pitch 143 and orientation φ of the corresponding 2D sinusoid pattern. The pitch 143 of the 2D sinusoid pattern 140 is determined by the radial distance r of the k-space point—more precisely, the pitch 143 is substantially the inverse of the radial distance r in the frequency domain. The orientation φ is the angle φ of the k-space points in a radial coordinate system in the frequency domain. Thus, a number of different excitation patterns may be generated by changing the pitch 143 of the 2D sinusoid pattern (or the angle (φ) between the two beams 131, 132) and changing the orientation φ of the 2D sinusoid pattern, with each different pair of pitch 143 and orientation φ of the 2D sinusoid pattern in the spatial domain corresponding to a different conjugate pair (radial distance r and orientation φ) in the k-space (frequency) domain.

Referring back to FIGS. 1A and 1B, the excited target 102 emits signals (or photons), and the signals are captured in optical imaging system 106 including an objective lens 124 and an imaging sensor (or imager) 126. The emitted signal will have a wavelength λ_{E}. The objective lens has magnification (Mag) and a numerical aperture NA=n×sinθ, where n is the index of refraction of the medium in which the lens 124 is placed and θ is the half-angle of the maximum cone of light that can enter or exit the lens 124. Typically, the imaging sensor 126 can be a charge-coupled device (CCD), complementary metal-oxide-semiconductor (CMOS) image sensor, or any other photon detectors in a matrix or array format including a plurality of pixels m. Note that, in some applications, the emitted signals from the target 102 may be directly captured by the imager 126 without going through the objective lens 124.

Then, it is determined 108 whether the images corresponding to all the phases of the 2D sinusoid excitation pattern were obtained. If images corresponding to all the phases of the 2D sinusoid excitation pattern were not obtained in step 108, the excitation phase is changed 114 and steps 104, 106, 108 are repeated for the changed excitation phase. If images corresponding to all the phases of the 2D sinusoid excitation pattern were obtained in step 108, then it is determined 110 whether the images corresponding to all the 2D sinusoid selective excitation patterns were obtained. If images corresponding to all the 2D sinusoid selective excitation patterns were not obtained in step 110, the excitation pattern is changed by using a different spatial frequency (e.g., changing the pitch 143 and the orientation φ of the 2D sinusoid pattern) and steps 104, 106, 108, 114 are repeated for the next selective excitation pattern.

If images corresponding to all the 2D sinusoid selective excitation patterns were obtained in step 110, then finally the captured images are sent to a computer for SAO post processing 112 and visualization. In conventional imaging, the resolution of the SAO imaging system is determined by the numerical aperture NA of the lens 124, the wavelength λ_{E} of the emitted light, and the pixel size. In contrast, in SAO imaging, the resolution of the imaging system is beyond what can be achieved by the numerical aperture NA of the lens 124, the wavelength λ_{E} of the emitted light, and the pixel size. Thus, as shown in FIG. 1B, the images captured through steps 104, 106, 108 of FIG. 1A are raw images RIᵢ with a resolution lower than (insufficient for) the resolution needed to resolve the objects on the imaging target 102. However, multiple sets of the lower resolution raw images RIᵢ are captured for different excitation phases and spatial frequencies (excitation patterns) to obtain the complete raw image set 128, which then goes through SAO post-processing 112 to synthesize the final image FI that has a resolution higher than the resolution of the raw images RIᵢ. The resolution of the final image FI obtained by SAO post-processing is sufficient for resolution of the objects on the imaging target 102. The methodology for SAO post-processing 112 for synthesizing high resolution images FI from lower resolution raw images RIᵢ is well known. Raw images RIi are converted into k-space information of the high resolution images FI, and this information is Fourier transformed to synthesize or reconstruct the high resolution images FI. For example, one example of the SAO post-processing methodology can be found in U.S. Pat. No. 6,016,196, issued on Jan. 18, 2000 to Mermelstein, entitled “Multiple Beam Pair Optical Imaging,” which is incorporated by reference herein.

Applying SAO to DNA (deoxyribonucleic acid) or RNA (ribonucleic acid) sequencing presents a number of challenges. The term “nucleic acid” herein includes both DNA and RNA. In DNA or RNA sequencing, single molecule or amplified clones of a DNA template (collectively referred to as “microparticle”) are immobilized onto a planar substrate. The array of microparticles then goes through multiple cycles of chemical reaction and optical detection. FIGS. 2A, 2B, and 2C illustrate different types of individual sequencing microparticles that can be used for DNA sequencing. FIG. 2A illustrates an individual microparticle 202 formed by a 1-micrometer diameter bead 208 covered with clonal DNA molecules 210 that have been previously amplified by a water-in-oil emulsion PCR technique. The bead 208 is attached directly to the substrate 204 in fluid 206. FIG. 2B illustrates an individual microparticle 202 as a cluster of clonal DNA molecules 210 attached to the substrate 204 and placed in fluid 206. The DNA molecules 210 have been previously amplified by a bridge amplification technique. FIG. 2C illustrates an individual microparticle as a single DNA molecule 210 attached to the substrate 204 and placed in fluid 206. The single DNA molecule 210 is sequenced without amplification.

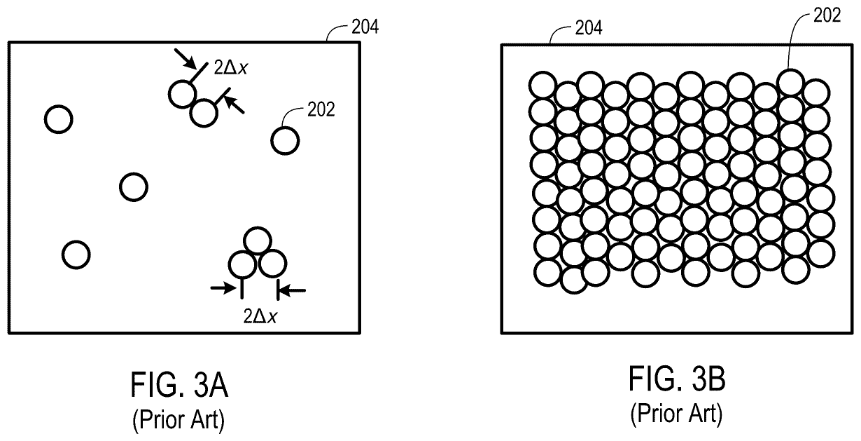

The distribution of DNA microparticles can be random or regular. FIGS. 3A and 3B illustrate some examples of the distribution of DNA microparticles. If Δx is defined to be the spatial resolution of an imaging system (i.e., Δx is the minimum distance of two point objects that can be resolved by the imaging system), Δx is typically designed to be about half of the distance between adjacent microparticles 202 (see FIG. 3A). In DNA sequencing applications, it is highly desirable for an optical imaging system to achieve both high resolution and high scanning speed at the same time. SAO imaging is promising since it can image a large area using a low magnification lens and camera without sacrificing resolution. The resolution of SAO imaging is obtained from the high resolution illumination patterns and post-processing. However, SAO requires selective excitation to be repeated for a number of selective excitation patterns. Conventional SAO imaging uses a large number of SAO excitation patterns, often including many redundant or even irrelevant illumination patterns. The number of excitation patterns in conventional SAO is merely determined based on the hardware architecture of the illumination system, without regard to other factors. The large number of excitation patterns in conventional SAO makes it impractical for use in DNA sequencing, as conventional SAO does not offer the cost and throughput benefit in DNA sequencing compared to conventional optics. Also, conventional SAO hardware is large, complex, difficult to scale, and mechanically and thermally unstable, requiring large space and extremely careful control of temperature and mechanical vibration for continuous run, making it particularly impractical for use in DNA sequencing which requires repeated, continuous runs of SAO over a very large number of DNA microparticle arrays.

Typically, given the final reconstructed image, a detection system estimates whether particles are present on a plurality of regions on a target by generating a set of reconstruction estimates based on the intensity values of the reconstructed image. For example, a reconstruction estimate for a pixel location of the reconstructed image may indicate whether a particle is present at a corresponding region of the target by comparing the intensity value for the pixel location to a predetermined threshold. However, it is often difficult to detect particles with high accuracy in this manner due to, for example, the texture of the target that results in a noisy reconstruction image.

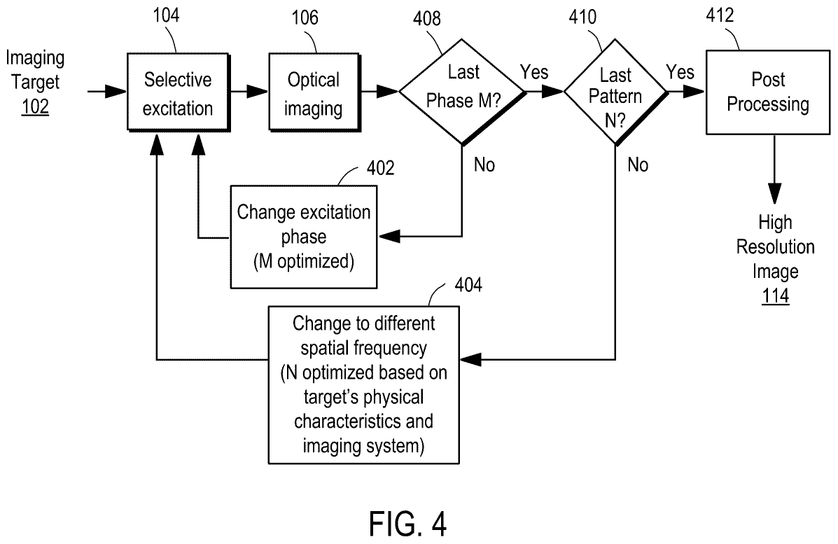

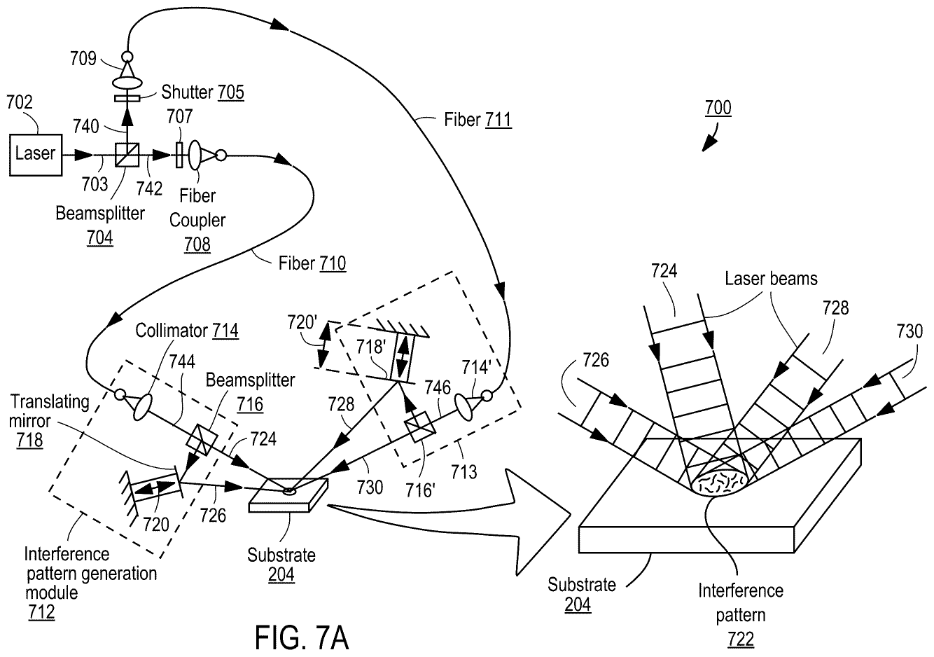

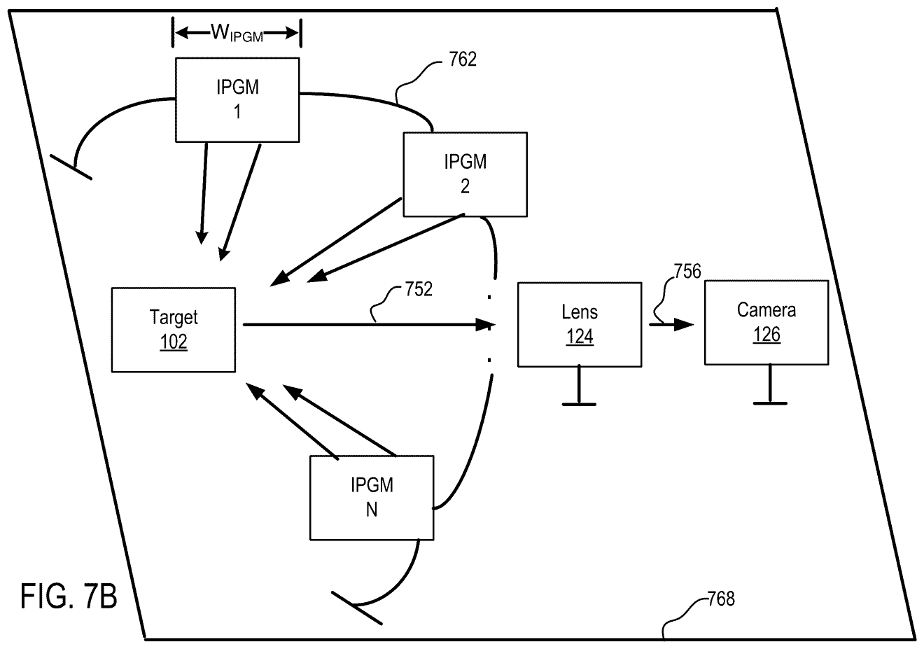

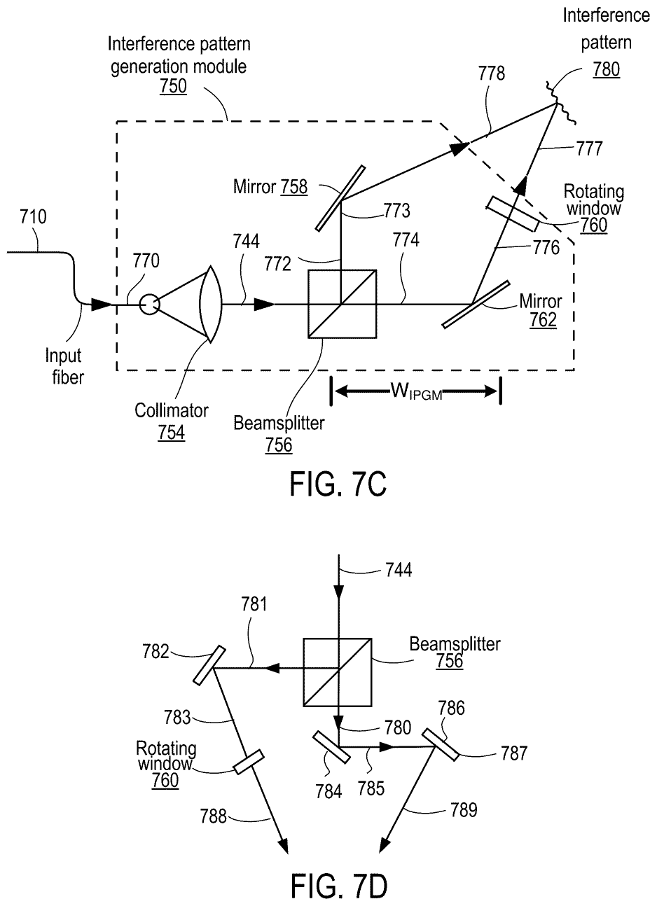

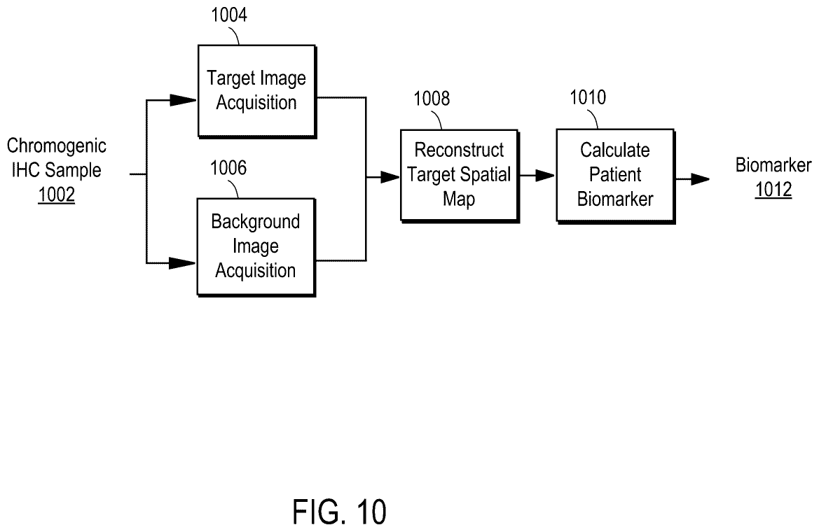

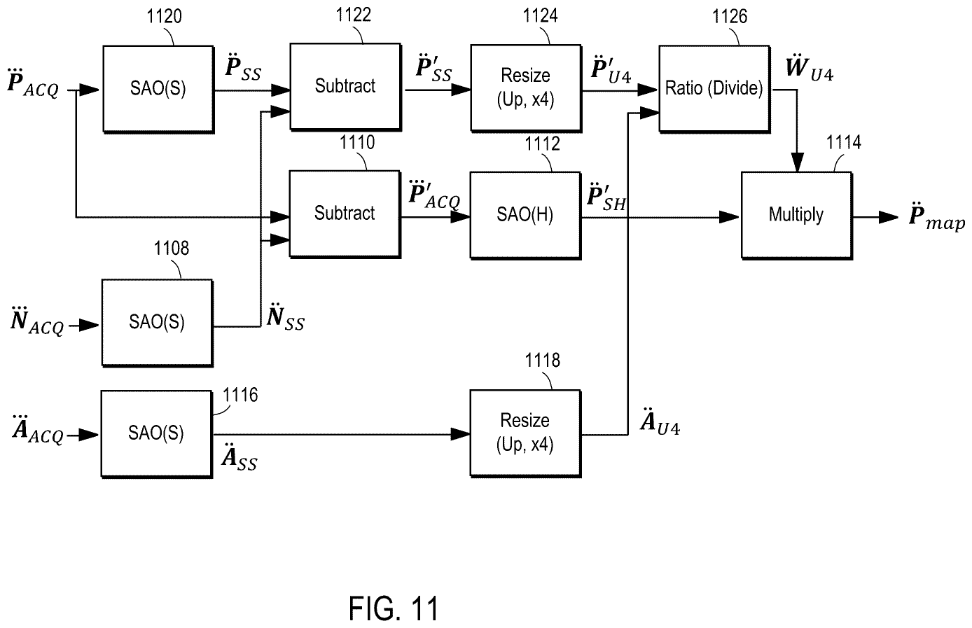

Turning to the figures, FIG. 4 illustrates an SAO method, according to one embodiment. As is typical with SAO imaging, selective excitation (or illumination) 104 is applied to an imaging target 102, and the light scattered or fluoresced from the imaging target 102 is captured by optical imaging 106. Here, the imaging target 102 is assumed to be a DNA microparticle such as those illustrated in FIGS. 2A-2C, 3A, and 3B, mRNA (messenger RNA) segments, lncRNA (long no-coding RNA), or proteins that appear as spots or particles when fluorescently labeled and imaged such as those illustrated in FIGS. 10-11, but is not limited hereto. As will be explained in more detail below with reference to FIGS. 7A-7D, selective excitation 104 is applied to the imaging target 102 by an illumination apparatus that is configured to cause interference of two light beams on the imaging target 102. The excited target 102 emits signals (or photons), and the emitted signals are captured in an optical imaging system 106 including an objective lens and an imaging sensor (or imager). Then, it is determined 408 whether the images corresponding to all M phases of the 2D sinusoid excitation pattern were obtained. If images corresponding to all the phases of the 2D sinusoid excitation pattern were not obtained in step 408, the excitation phase is changed 402 and steps 104, 106, 408 are repeated for the changed excitation phase. If images corresponding to all the phases of the 2D sinusoid excitation pattern were obtained in step 408, then it is determined 410 whether the images corresponding to all the 2D sinusoid selective excitation patterns were obtained. If images corresponding to all the 2D sinusoid excitation patterns were not obtained in step 410, the excitation pattern is changed by using a different spatial frequency (e.g., changing the pitch 143 and the orientation φ of the 2D sinusoid pattern) and steps 104, 106, 408, 402, 410, 404 are repeated for the next selective excitation pattern. Then, if images corresponding to all the 2D sinusoid excitation patterns were obtained in step 410, then the captured images are sent to a computer for SAO post processing 412 and visualization to obtain the high-resolution images 114 of the imaging target 102 from the captured lower resolution raw images. As explained above, the raw images captured by optical imaging 106 have a resolution insufficient to resolve the objects on the imaging target 102, while the high resolution image 114 reconstructed by SAO post-processing 412 have a resolution sufficient to resolve the objects on the imaging target 102.

The SAO method of the present invention uses an optimized number N of selective excitation patterns and an optimized number M of excitation phases of each selective excitation pattern, so that SAO can be used to image targets such as DNA microparticles in a massively parallel manner within a short amount of time. As explained above, the number of selective excitation patterns used in conventional SAO is determined merely by the hardware characteristics of the illumination system, independent and without consideration of the imaging target or the imaging system (objective lens and camera). Thus, the number of k-space sampling points corresponding to the selective excitation patterns in conventional SAO was not optimized, and has many redundant and sometimes irrelevant k-space sampling points. In contrast, SAO according to the embodiments of the present invention herein uses selective excitation patterns whose number N is optimized and minimized as a function of the imaging target's physical characteristics corresponding to spatial frequency content (e.g., the size, shape, and/or spacing of the objects on the imaging target). SAO according to the embodiments herein may also use selective excitation patterns whose number N is optimized alternatively or additionally as a function of various parameters of the imaging system (e.g., magnification (Mag) of the objective lens, numerical aperture (NA) of the objective lens, wavelength λ_{E} of the light emitted from the imaging target, and/or effective pixel size p of the pixel sensitive area of the CCD, etc.). In this manner, the resulting number N of excitation patterns used in SAO becomes much smaller than that in conventional SAO, thereby enabling SAO to be used with DNA sequencing that requires massive parallelization of SAO imaging in a short amount of time to make DNA sequencing commercially feasible. Thus, dramatic reduction of cost and increase of throughput of DNA sequencing can be achieved.

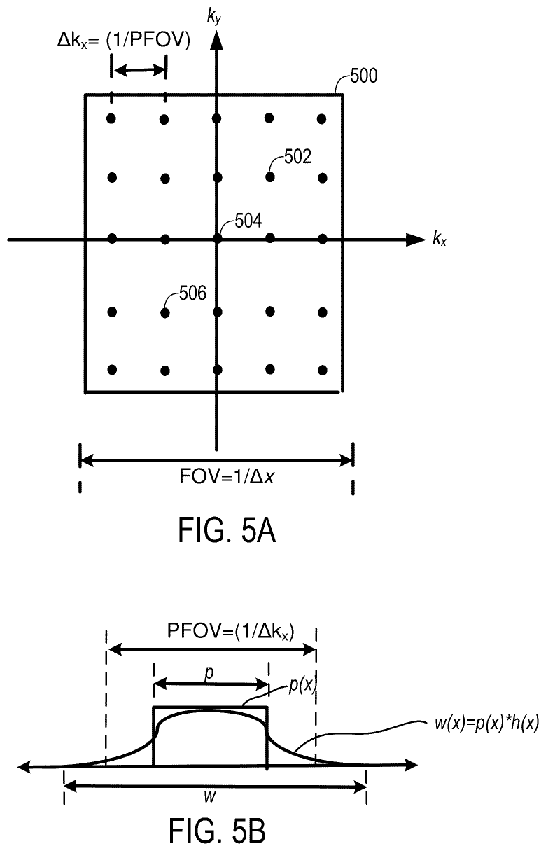

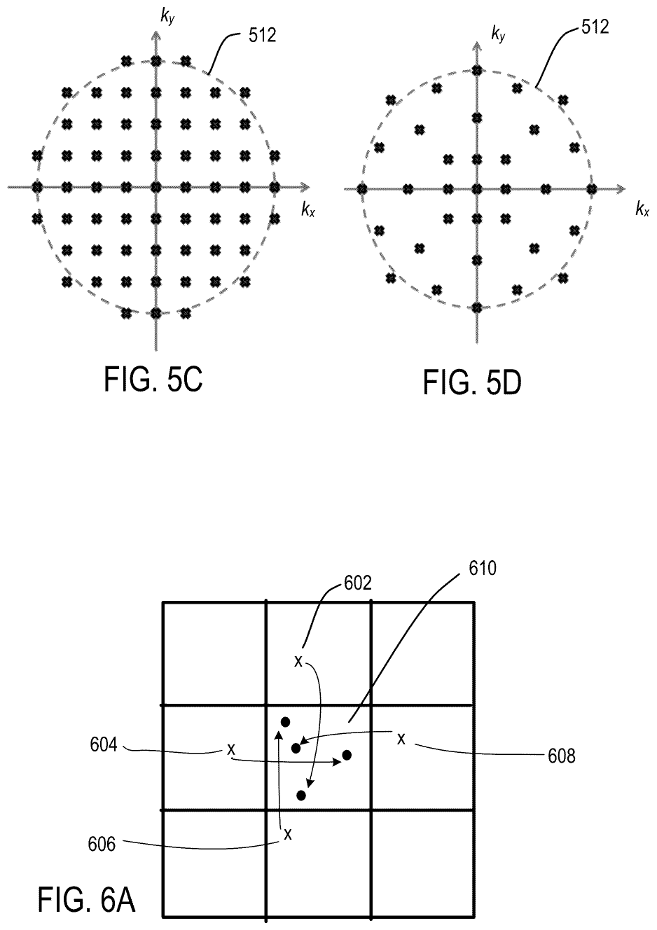

FIG. 5A illustrates the k-space sampling points (selective excitation patterns) used in SAO, according to one embodiment. In FIG. 5A, it is assumed that the CCD imaging area has a square shape and thus a square shaped k-space sampling space 500 for SAO is also assumed, although the description for FIG. 5A below can be applied to a non-square shaped (e.g., rectangular) k-space sampling space as well. The k-space sampling space 500 has an area of FOV², with the extent of the k-space sampling space in each of the horizontal and vertical directions being FOV. Here, FOV stands for the k-space field of view. In the k-space frequency domain, FOV should be equal to (1/Δx), where Δx the spatial resolution of an imaging system (i.e., Δx is the minimum distance of two point objects that can be resolved by the imaging system). Each conjugate pair 502, 506 and its DC point 504 correspond to one selective excitation pattern for SAO as used with the present invention. Thus, the number of selective excitation patterns used in SAO corresponds to the number of conjugate pairs of k-space points in the k-space sampling space 500 (FOV×FOV). Δkₓ is the k-space sampling interval, and is equal to (1/PFOV) where PFOV is the pixel field of view. The smaller the k-space sampling interval Δkₓ is in the k-space sampling space 500, the larger the number of k-space points and the corresponding number of excitation patterns are. Specifically, the following equations hold:

N=floor(L/2) (Equation 1),
where L is the number of k-space points in the k-space, N is the number of selective excitation patterns, and floor ( ) rounds the number to the nearest but smallest integer;

L=round((FOV/Δkₓ)²)=round ((PFOV/Δx)²) Equation 2),
where round ( ) rounds the number to the nearest integer, and PFOV is the extent in the reciprocal (or Fourier) space of the sampling space (k-space) to be reconstructed from the samples.

FIG. 5B illustrates the selection of the k-space sampling interval used in SAO, according to one embodiment. As explained above, the imaging target size determines the required spatial resolution Δx. Magnification (Mag) and CCD pixel size (Z) determines the effective pixel size p on the imaging-target plane, p=Z/Mag. As shown in FIG. 5B, the detected area w(x) (i.e., the area captured by the pixel) can be represented as the convolution of the pixel-sensitivity function p(x) (e.g., the rectangular function with width p) and the point-spread function (PSF) h(x) of the lens (e.g., a bell-shaped curve). The width w can be defined as the 1/e² width of detected area w(x). Since the PSF of the lens is determined by the NA of the lens, the extent of the detected area (w) and the weighting over the detected area (i.e., the effective sensitivity profile over the detected area) are the function of the magnification (Mag) of the lens, numerical aperture (NA) of the lens, and the CCD pixel size (Z).

As can be seen from the above, the k-space sampling space (FOV) is determined by the desired spatial resolution Δx and is dictated by the imaging target. The particles of interest, such as biological particles like DNA microparticles or mRNA segments typically have a very small size, resulting in a large k-space sampling space. In conventional SAO, the k-space sampling interval Δkₓ is set without regard to the physical characteristics of the imaging target or the parameters of the imaging system, and is rather just set randomly according to whatever interval allowed by the SAO illumination system. This made the number of k-space points and the resulting selective excitation patterns prohibitively large for use in DNA sequencing applications using SAO, because of the high cost and low throughput of DNA sequencing using such large number of selective excitation patterns in SAO.

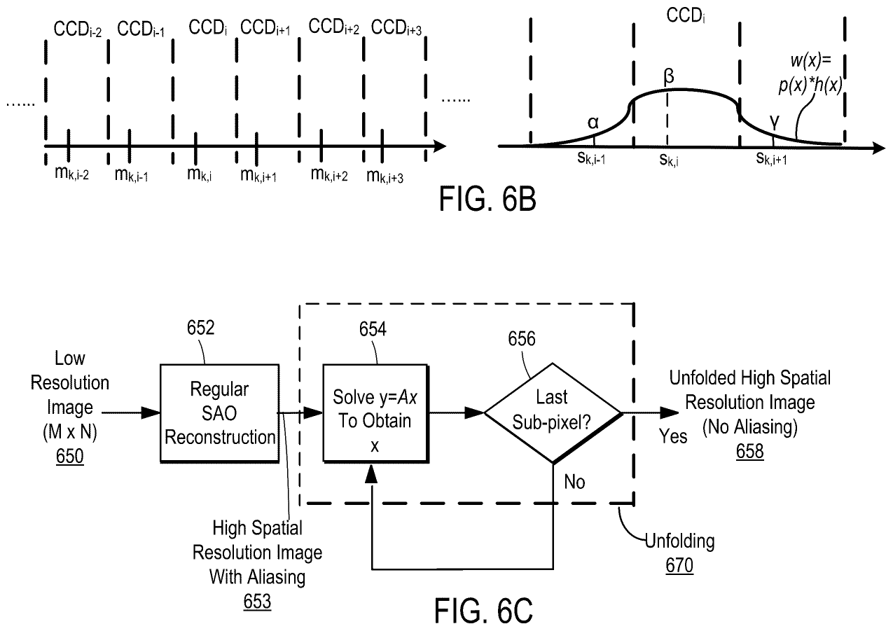

In contrast, SAO according to the embodiments of the present invention herein use selective excitation patterns whose number N is optimized as a function of the imaging target's physical characteristics corresponding to spatial frequency content (e.g., the size, shape, and/or spacing of the imaging target). As shown in FIG. 5B, in one embodiment, the pixel field of view PFOV is selected to be smaller than the extent (w) of the detected area, i.e., PFOV<w. Using a small PFOV results in a larger k-space sampling interval Δkₓ, thereby reducing the number (L) of k-space points in the k-space sampling space 500 and the resulting number (N) of selective excitation patterns for use in SAO. As will be explained in more detail below with reference to FIGS. 6A and 6B, using PFOV smaller than the extent (w) of the detected area causes aliasing in the high resolution image obtained from SAO, but such aliasing can be removed using the method as described below with reference to FIG. 6C. In other embodiments, the PFOV may be set to be equal to or larger than the extent (w) of the detected area, thereby preventing aliasing from occurring in the high resolution image obtained from SAO. Also note that setting PFOV with consideration of the extent (w) of the detected area effectively sets the k-space sampling interval (Δkₓ) and the resulting number (N) of selective excitation patterns based on the various parameters of the imaging system, since the extent (w) of the detected area is a function of the magnification (Mag) of the lens, numerical aperture (NA) of the lens, and the CCD pixel size (Z) as explained above.

Furthermore, SAO according to the embodiments herein further reduces the number of iterations of selective excitation and imaging by minimizing the number of phase changes (M in steps 402, 408 of FIG. 4). Referring back to FIG. 5A and as explained above, one conjugate pair 502, 506 of k-space points corresponds to one SAO interference pattern generation module that produces a specific pitch and orientation of one selective excitation pattern. The DC point 504 corresponds to the signal offset of the 2D sinusoid selective excitation pattern. Thus, in one embodiment, three different measurements at three different phases of the interference pattern with the same pitch and orientation are made to distinguish between the two conjugate points 502, 506 and the DC point 504 in the k-space. This is in contrast to conventional SAO, where more than three phases were used to illuminate and image each selective excitation pattern for SAO. In another embodiment, since the DC point 504 is common for all conjugate pairs 502, 506, it is also possible to utilize the DC point 504 obtained in one 2D sinusoid pattern with a specific pitch and orientation to obviate the need for illuminating and imaging the selective excitation pattern at the DC point 504 of another selective excitation pattern with a different pitch and orientation, thereby reducing the number M of changed phases needed for imaging in steps 402, 408 (FIG. 4) to two (2) phases for the other selective excitation patterns. In other words, each interference pattern generation module produces a pattern with only two different phases, except one module that produces pattern with three different phases to acquire the DC point 504. For optimal tolerance to noise, one can choose specific phases for the patterns. For three different phases per a specific selective excitation pattern, the optimal phase difference may be 0, 120, and 240 degrees. For two different phases per a specific selective excitation pattern, the optimal phase difference may be 0 and 90 degrees.

Since the objects of interest (i.e., biological particles such as DNA microparticles, mRNA segments, lncRNA, or proteins) are typically circularly symmetric, the k-space spectrum of the objects of interest will also be circularly symmetric and thus only k-space samples in the circular region with diameter of FOV (=1/Δx) may be needed for SAO. Thus, in one embodiment, the SAO according to the present invention uses selective excitation patterns corresponding to the k-space sampling points within the circular region 512, as shown in FIG. 5C.

FIG. 5D illustrates reducing the number of k-space sampling points by sparse k-space sampling, according to one embodiment. Conventional SAO methods do not utilize frequency information of the objects in the image scene. Solid objects such as beads used in microparticles have much less energy in the high spatial-frequencies compared to the low frequencies. Therefore, under-sampling in the high spatial frequencies is more tolerable than under-sampling in the low spatial frequency region. Thus, in one embodiment of the present invention, the number (N) of selective excitation patterns is further reduced by non-uniform or variable-density sampling in the Fourier space as shown in FIG. 5D. The penalty for not meeting the Nyquist sampling rate in high spatial frequencies is tolerable in SAO for DNA sequencing applications, and thus SAO according to the embodiments herein relaxes the Nyquist sampling criteria in the higher-frequencies, thereby reducing the number of selective excitation patterns by almost half of what would be required with uniform sampling. For example, the number of k-space samples in the embodiment of FIG. 5D is only 54% of the number of k-space samples in the embodiment of FIG. 5C.

FIG. 6A illustrates how aliasing occurs in SAO by use of a pixel field of view smaller than the detected area, according to one embodiment. As mentioned above with reference to FIG. 5B, using a pixel field of view (PFOV) smaller than the extent (w) of the detected area, i.e., PFOV<w, results in aliasing in the image obtained for SAO, because each pixel in the CCD would detect areas larger than the pixel itself. The extra area (i.e., left and right parts of the extent (w) outside of p(x)) is the area also detected by its neighboring pixels in the CCD. This is illustrated in FIG. 6A, where the objects 602, 604, 606, 608 detected in the extra area in the neighboring pixels will enter into the center pixel 610 (assuming rectilinear sampling in the k-space), resulting in aliasing and unwanted artifacts that degrade the image quality.

FIG. 6B illustrates how the actual signal at a pixel of an imaging system may be determined by unfolding the measured signal at the pixel to remove aliasing, according to one embodiment. In order to remove aliasing in the measured image signal and obtain the actual image signals, one can formulate a linear equation in the form of y=Ax at a particular sub-pixel k at pixel CCDᵢ of the CCD. Referring to FIG. 6B, m_{k,i} represents the measured signal (including aliasing) at a particular k-th sub-pixel location within the i-th CCD pixel CCDᵢ. Note that the relative locations of all measured signals m_{k,i} (i=1, . . . , ∞) within their i-th CCD pixels are the same. s_{k,i} represents the actual or ideal signal of the object at the k-th sub-pixel locations within the i-th CCD pixel CCDᵢ. α, β, and γ represent the values of the weighting function w(x) of the i-th CCD pixel at the locations corresponding to s_{k,i-1}, s_{k,i}, and s_{k,i+1}, respectively, and s_{k,i} (i=1, . . . , ∞) is the actual (ideal) signal at the particular k-th sub-pixel location within the i-th CCD pixel CCDᵢ. As explained above, the weighting function w(x) can be represented as the convolution of the pixel-sensitivity function p(x) (e.g., the rectangular function with width p) and the point-spread function (PSF) h(x) of the lens (e.g., a bell-shaped curve). With these parameters defined and assuming that the number of pixels of the CCD is infinite, one can write the signal-equation series for a particular k^{th} sub-pixel location as a linear matrix equation y=Ax, where y=[m_{k,1}, m_{k,2}, . . . ], x=[s_{k,1}, s_{k,2}, . . . ], and A is a matrix with elements being zeros and values of the weighting function (e.g., α, β, and γ). The linear matrix equation y=Ax shows that the “unfolding” process (i.e., recovering the actual signal s_{k,i}) can be viewed as a common inverse problem of y=Ax (i.e., x=A⁻¹y). In other embodiments, if non-rectilinear sampling pattern is used (e.g., variable-density, radial sampling, etc.), the actual relationship between sᵢ and mᵢ will change from that shown in FIG. 6B, in which case the point-spread-function (i.e., impulse response) can be measured in either simulation or real experiments to construct the inversion matrix (A⁻¹).

FIG. 6C illustrates a method of unfolding the measured signal at the pixel to remove aliasing, according to one embodiment. The steps 652, 654, 656 together constitute the post-processing steps for SAO. In conventional SAO, post-processing includes only the regular SAO reconstruction 652 to generate the high spatial resolution image 653 from the low resolution images (M×N) 650 obtained from selective excitation of the imaging target. However, in SAO according to the embodiments of the present invention herein, post-processing includes the “unfolding” step 670 to remove aliasing from the high spatial resolution image 653 that contains aliasing resulting from using PFOV smaller than the extent (w) of the detected area for selective excitation. The unfolding process 670 includes solving the linear equation y=Ax to recover the actual signals x, which is repeated 656 at each sub-pixel location, for all CCD pixels. As a result, a high spatial resolution image 658 without aliasing can be obtained from SAO, despite using PFOV smaller than the extent (w) of the detected area for selective excitation in SAO.

Note that “unfolding” as explained herein can also be used to improve the SAO image reconstruction quality even when PFOV larger than or equal to the extent (w) of the detected area is used for selective excitation in SAO. In conventional SAO, the reconstructed pixels are simply cropped (to the width being p) and stitched together. This way of “crop and stitch” still does not undo the apodization caused by the weighting function w(x). In contrast, “unfolding” may be used according to the present invention even when PFOV larger than or equal to the extent (w) of the detected area is used for selective excitation in SAO such that no aliasing occurs. Since the unfold process is fundamentally undoing (i.e., unapodizing) the weighting function w(x), the “unfold” process can also be used to improve image reconstruction even when PFOV>=w is used for SAO selective excitation.

FIG. 7A illustrates a structured illumination apparatus for selectively exciting the microparticles, according to one embodiment. The illumination apparatus shown in FIG. 7A is merely exemplary, and various modifications may be made to the configuration of the illumination apparatus for SAO according to the present invention. The example illumination apparatus in FIG. 7A shows only two interference pattern generation modules (IPGM) 712, 713 for simplicity of illustration, but for biological sequencing applications such as real DNA sequencing applications, mRNA sequencing applications, or lncRNA sequencing applications there would be a larger number of IPGMs. Each IPGM is in modular form and is configured to generate one selective excitation pattern at a given pitch and orientation, corresponding to one conjugate pair of the k-space sampling points. Thus, there is a one-to-one relationship between an IPGM and a 2-D sinusoid selective excitation pattern at a given pitch and orientation and to one conjugate pair of the k-space sampling points. A larger number (N) of selective excitation patterns would require a larger number of IPGMs in the SAO illumination apparatus.

The structured illumination apparatus 700 generates multiple mutually-coherent laser beams, the interference of which produces interference patterns. Such interference patterns are projected onto the microparticle array substrate 204 and selectively excite the DNA microparticles 202. Using the interference of multiple laser beams to generate the interference patterns is advantageous for many reasons. For example, this enables high-resolution excitation patterns with extremely large FOV and DOF. Although the structured illumination apparatus of FIG. 7A is described herein with the example of generating excitation patterns for DNA microparticles, it should be noted that the structured illumination apparatus of FIG. 7A can be used for any other type of application to generate excitation patterns for imaging any other type of target, such as biological particles including DNA segments, mRNA (messenger RNA) segments, lncRNA (long no-coding RNA), or proteins that appear as spots or particles when fluorescently labeled and imaged. Examples of imaging mRNA segments using selective excitation patterns are described below in conjunction with FIGS. 10-11.

Referring to FIG. 7A, the structured illumination apparatus 700 includes a laser 702, a beam splitter 704, shutters 705, 707, fiber couplers 708, 709, a pair of optical fibers 710, 711, and a pair of interference pattern generation modules (IPGMs) 712, 713. As explained above, each IPGM 712, 713 generates an interference pattern (selective excitation pattern) that corresponds to one conjugate pair of k-space sampling points. The beam 703 of the laser 702 is split by the beam splitter 704 into two beams 740, 742. A pair of high-speed shutters 705, 707 is used to switch each beam 740, 742 “on” or “off” respectively, or to modulate the amplitude of each beam 740, 742, respectively. Such switched laser beams are coupled into a pair of polarization-maintaining optical fibers 711, 710 via fiber couplers 709, 708. Each fiber 711, 710 is connected to a corresponding interference pattern generation module 713, 712, respectively. The interference pattern generation module 713 includes a collimating lens 714′, a beam splitter 716′, and a translating mirror 718′, and likewise the interference pattern generation module 712 includes a collimating lens 714, a beam splitter 716, and a translating mirror 718.

The beam 744 from the optical fiber 710 is collimated by the collimating lens 714 and split into two beams 724, 726 by the beam splitter 716. The mirror 718 is translated by an actuator 720 to vary the optical path-length of the beam 726. Thus, an interference pattern 722 is generated on the substrate 204 in the region of overlap between the two laser beams 724, 726, with the phase of the pattern changed by varying the optical path-length of one of the beams 726 (i.e., by modulating the optical phase of the beam 726 by use of the translating mirror 718).

Similarly, the beam 746 from the optical fiber 711 is collimated by the collimating lens 714′ and split into two beams 728, 730 by the beam splitter 716′. The mirror 718′ is translated by an actuator 720′ to vary the optical path-length of the beam 728. Thus, the interference pattern 722 is generated on the substrate 204 in the region of overlap between the two laser beams 728, 730, with the pattern changed by varying the optical path-length of one of the beams 728 (i.e., by modulating the optical phase of the beam 728 by use of the translating mirror 718′).

As shown in FIG. 7A, each IPGM 712, 713 is implemented in modular form according to the embodiments herein, and one IPGM produces an interference pattern corresponding to one conjugate pair of k-space points. This modularized one-to-one relationship between the IPGM and the k-space points greatly simplifies the hardware design process for SAO according to the embodiments herein. As the number of selective excitation patterns used for SAO is increased or decreased, the SAO hardware is simply changed by increasing or decreasing the number of IPGMs in a modular manner. In contrast, conventional SAO apparatuses did not have discrete interference pattern generation modules but had a series of split beams producing as many multiple interferences as possible. Such conventional way of designing SAO apparatuses produced non-optimized or redundant patterns, slowing down and complicating the operation of the SAO system.

While this implementation illustrated in FIG. 7A is used for its simplicity, various other approaches can be used within the scope of the present invention. For example, the amplitude, polarization, direction, and wavelength, in addition to or instead of the optical amplitude and phase, of one or more of the beams 724, 726, 728, 730 can be modulated to change the excitation pattern 722. Also, the structured illumination can be simply translated with respect to the microparticle array to change the excitation pattern. Similarly, the microparticle array can be translated with respect to the structured illumination to change the excitation pattern. Also, various types of optical modulators can be used in addition to or instead of the translating mirrors 718, 718′, such as acousto-optic modulators, electro-optic modulators, a rotating window modulated by a galvanometer and micro-electro-mechanical systems (MEMS) modulators. In addition, although the structured illumination apparatus of FIG. 7A is described herein as using a laser 702 as the illumination source for coherent electro-magnetic radiation, other types of coherent electro-magnetic radiation sources such as an SLD (super-luminescent diode) may be used in place of the laser 702.

Also, although FIG. 7A illustrates use of four beams 724, 726, 728, 730 to generate the interference pattern 722, larger number of laser beams can be used by splitting the source laser beam into more than two beams. For example, 64 beams may be used to generate the interference pattern 722. In addition, the beam combinations do not need to be restricted to pair-wise combinations. For example, three beams 724, 726, 728, or three beams 724, 726, 730, or three beams 724, 728, 730, or three beams 726, 729, 730, or all four beams 724, 726, 728, 730 can be used to generate the interference pattern 722. Typically, a minimal set of beam combinations (two beams) is chosen as necessary to maximize speed. Also, the beams can be collimated, converging, or diverging. Although different from the specific implementations of FIG. 7A and for different applications, additional general background information on generating interference patterns using multiple beam pairs can be found in (i) U.S. Pat. No. 6,016,196, issued on Jan. 18, 2000 to Mermelstein, entitled “Multiple Beam Pair Optical Imaging,” (ii) U.S. Pat. No. 6,140,660, issued on Oct. 31, 2000 to Mermelstein, entitled “Optical Synthetic Aperture Array,” and (iii) U.S. Pat. No. 6,548,820, issued on Apr. 15, 2003 to Mermelstein, entitled “Optical Synthetic Aperture Array,” all of which are incorporated by reference herein.

FIG. 7B illustrates the arrangement of the illumination pattern generation modules in a half ring structure according to one embodiment. Referring to FIG. 7B, multiple IPGMs (IPGM 1, IPGM 2, . . . , IPGM N) such as IPGMs 712, 713 (FIG. 7A) are arranged substantially symmetrically in a half-ring shape on a half-ring shaped, monolithic structure 762, to generate the selective excitation patterns. The half-ring structure 762 is fixed on the system table 768. In the embodiment of FIG. 7B, the N IPGMs generate N selective excitation patterns for SAO on the imaging target 102, and the scattered or fluoresced light 752 is passed through objective lens 124 and captured 756 by camera 126 which may be a CCD camera.

These arrangements of the IPGMs in the embodiment of FIG. 7B enable a monolithic and compact holding structure that has multiple benefits for enabling the SAO system to be used for DNA sequencing applications, compared to conventional optical-bench SAO systems where each optical component is individually mounted on its holding structure. The monolithic structure 762 enables the IPGM arrangement to be compact and symmetric, and this compact, symmetric, and monolithic structure preserves more stable channel-to-channel and beam-to-beam geometry against mechanical and thermal distortions. The compact monolithic structure 762 is also less susceptible to non-flatness or torsional and bending modes of the optical table 768, and the symmetric arrangement of the IPGMs around the half-ring structure 762 makes the effect of heat contraction or expansion less detrimental to the beam geometry, i.e., the channel-to-channel or beam-to-beam angles of laser beams are changed less compared to a non-symmetric structure. Furthermore, the compact design shortens the travel distances of the laser beam in air, making it easy to prevent air disturbances affecting the stability of the interference pattern that may cause the effective optical path length to change resulting in change of the interference fringe position. Such stability allows more accurate calibration of the beam geometry. Furthermore, the half-ring arrangement of the IPGMs in FIG. 7B has the additional advantage that it enables the imaging module (i.e., camera 126 and objective lens 124), illumination structure (i.e., the half-ring 762), and the imaging target 102 to be placed on one stiff structure (e.g., optical table) 768.

FIG. 7C illustrates the internal structure of an illumination pattern generation module, according to one embodiment. The embodiment of FIG. 7C has a rotating window 760 in IPGM 750 that is placed after the mirror 762. The beam 770 from the optical fiber 710 is collimated by the collimating lens 754 and the collimated beam 744 is split into two beams 773, 774 by the beam splitter 756. Beam 773 is reflected by mirror 758 and the reflected beam 778 is projected onto the imaging target to generate the interference pattern 780. Beam 774 is reflected by mirror 762 and the optical path-length of the reflected beam 776 is modulated by optical window 760 that is rotated, using a galvanometer, thereby modulating the optical phase of the corresponding beam 776 and generating a modulated beam 777. The interference pattern 780 is generated in the region of overlap between the two laser beams 777, 778, with the pattern changed by varying the optical path-length of one of the beams 777. By placing the rotating window 760 after the mirror 762, the width W_{IPGM} and the size of IPGM 750 can be reduced, as compared to the embodiment of FIG. 7A and FIG. 7D illustrated below. Thus, the half-ring shaped structure 762 holding the IPGMs can be made more compact, since the width W_{IPGM} of the IPGM directly affects the radius of the half-ring, for example, as shown in FIG. 7B.

FIG. 7D illustrates the internal structure of an illumination pattern generation module, according to another embodiment. IPGMs in the embodiments of FIGS. 7A and 7C may produce two beams that do not have equal path length between the interfering point at the imaging target and the splitting point (i.e., the beam splitter). The non-equal path length may significantly reduce the sinusoidal contrast if a relatively short coherent-length laser is used and also limit the applicability of the SAO system to only a specific wavelength (e.g., 532 nm green laser) since only a small number of lasers with specific wavelengths have a sufficiently long coherent-length that can be used with such non-equal-path IPGMs for good sinusoidal contrast. Compared to the embodiment of FIG. 7A, the embodiment of FIG. 7D uses additional folding mirrors to achieve equal paths between the two split beams. The laser beam 744 is split into beams 781, 780 by beam splitter 756. Beam 781 is reflected by mirror 782 and its optical path-length is modulated by rotating window 760 to generate beam 788. On the other hand, beam 780 is reflected twice by two mirrors 784, 787 to generate the reflected beam 789. Beam 788 and 789 eventually interfere at the imaging target to generate the selective excitation patterns. By use of two mirrors 784, 786, the optical path 744-780-785-789 is configured to have a length substantially equal to the length of the optical path 781-783-788. This equal-path scheme allows lasers with short coherent lengths to be used to generate interference patterns with high contrast. Moreover, this equal-path scheme enables the SAO system to be used with wavelengths other than 532 nm, thus making multiple-color SAO practical.

### Digital Pathology-Based Biomarker Detection Using Fluorescent Image Analysis of Chromogenic IHC Stains

In one embodiment, the system and methods described herein generates a high-resolution spatial map of a target by SAO imaging. In one embodiment, the target being imaged herein is a chromogenic IHC-stained sample. Thus, the techniques described herein may be applied to any existing IHC-stained samples to obtain the high-resolution target spatial map, without having to provide additional materials or configurations. In one embodiment, the target is antigen(s) in a tissue sample. However, it is appreciated that the target can be DNA, RNA, or any other biological body.

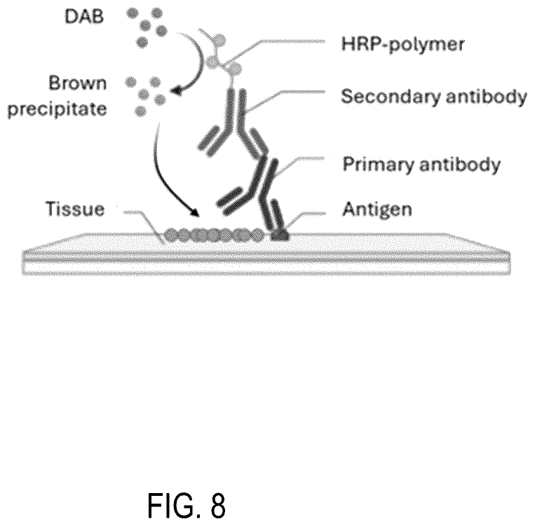

FIG. 8 illustrates labeling method of a target via chromogenic IHC staining, according to one embodiment. In one embodiment, a method is performed to specifically label the target via chromogenic IHC staining. The tissue sample is fixed using formalin, embedded in in paraffin wax, sliced into thin sections and placed on a microscope slide. The slide is washed in xylene and alcohol to remove the wax and rehydrate the tissue. Heat-induced epitope retrieval (HIER) or enzyme-induced retrieval is performed to break the protein cross-links and to expose the target. Natural enzymes (like peroxidase) and nonspecific binding are blocked to reduce background staining.

As illustrated in FIG. 8, a primary antibody that specifically binds to the target antigen is applied. A secondary antibody that binds to the primary antibody and is linked to an enzyme (e.g., HRP) is applied. A chromogenic solution (e.g., DAB) is added. The chromogenic solution reacts with the enzyme and creates a colored spot where the target protein is located. In one instance, counterstain (e.g., hematoxylin) is added to create colored spots where nuclei are located. In one instance, no fluorescent dyes are used in the chromogenic IHC process. Counterstain (e.g., hematoxylin) can be added to visualize cell nuclei. In one instance, no fluorescent dyes are used in the chromogenic IHC process, allowing conventional IHC-stained samples to be reused for fluorescent image acquisition.

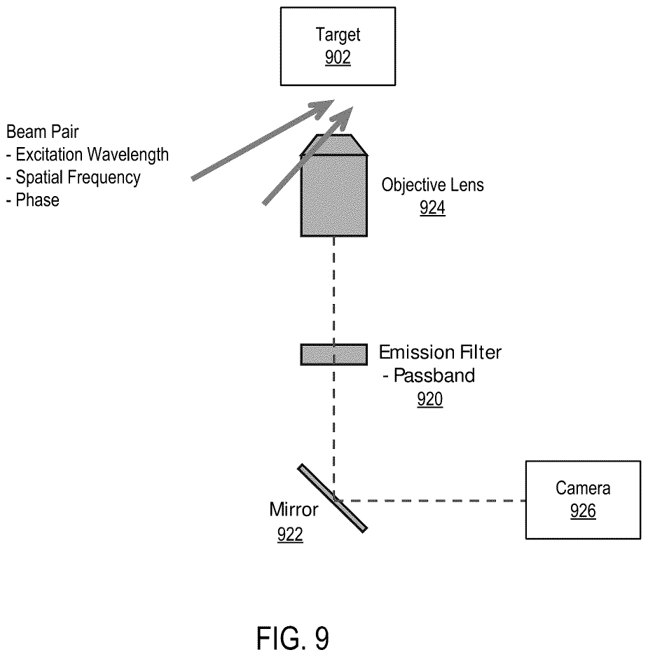

FIG. 9 illustrates an acquisition system 900 for acquiring raw images from a target, according to one embodiment. In one embodiment, the methods described below acquire one or more raw images from the target (e.g., proteins in IHC sample) 902 that are used to obtain a reconstructed image at a higher resolution. In one embodiment, the acquisition system 900 includes a set of IPGMs (not shown in FIG. 9 for the sake of simplicity), similar to that described with respect to FIG. 7B. As described in detail above, each IPGM corresponds to one selective excitation pattern for a given spatial frequency at a given pitch and orientation. In a primary example, there are N=4 different 2D excitation patterns and thus, 4 IPGMs.

In one embodiment, each IPGM for a given spatial frequency is configured to generate a beam pair with the selective excitation pattern with the spatial frequency, and also a phase and an excitation wavelength. In one instance, an IPGM can generate illuminations with M=3 different phases for a given spatial frequency. The phase can be controlled by a rotating window as described above. In one instance, an IPGM can generate 4 different excitation wavelengths of light. However, this is simply an example, and it is appreciated that each IPGM can generate illuminations with an additional number of phases and/or an additional number of excitation wavelengths.

As illustrated in FIG. 9, when the target 902 is illuminated using a beam pair with an excitation wavelength, a spatial frequency, and a phase, an objective lens 924 captures signals from the illuminated target, and passes the signals to an emission filter 920. An emission filter 920 passes signals within a specific range of wavelengths. In one instance, there are 10 different emission filters that each have a respective passband, controlled by a filter wheel. The filtered signals are reflected on a mirror 922 and sent to a camera 926, where a raw image is obtained for the structured illumination.

As described below, an acquisition channel may be defined by a combination of a particular excitation wavelength (in the illumination) and emission filter pair (for signal detection). Different acquisition channels are used to detect signals for different types of biological molecules. Therefore, in the example, the set of IPGMs may generate N×M=12 different raw images for a given acquisition channel.

FIG. 10 illustrates a target spatial map imaging method using SAO, according to another embodiment. Some or all of the steps in FIG. 10 can be performed by a computing system. In one embodiment, a background image is acquired 1006 by illuminating a first series of structured illuminations. In one instance, the first series of structured illuminations are obtained using a first acquisition channel. For example, the excitation wavelengths in the first acquisition channel are shorter than 550 nm. A lens and a camera are used to capture a series of raw background images of the labeled target, similarly to that described in FIG. 7A or 9. An emission filter of the first acquisition channel is used to selectively pass the light from the labeled target within a predefined wavelength range.

In one embodiment, a target image is acquired 1004 by illuminating a second series of structured illuminations. In one instance, the second series of structured illuminations are obtained using a second acquisition channel that is different from the first acquisition channel with respect to the excitation wavelength and/or emission filter value. For example, the excitation wavelengths in the second acquisition channel are longer than 600 nm. A lens and a camera are used to capture a series of raw target images of the labeled sample, similarly to that described in FIG. 7A. An emission filter of the second acquisition channel is used to selectively pass the light from the labeled target within a predefined wavelength range.

The target spatial map is reconstructed 1008 to obtain the higher-resolution reconstructed image. In one embodiment, a computer program or artificial intelligence (AI) model processes the raw background images and the raw target images to reconstruct the spatial distribution map by at least applying SAO imaging to the raw intensity signals. The target special map indicates the abundance of the target at each spatial location at a resolution that is higher than what would be attainable by the specification of the lens and the camera.

In one embodiment, the biomarker data 1012 for the patient that the tissue sample is associated with can be calculated 1010 based on the high-resolution target spatial map.

FIG. 11 illustrates a target spatial map imaging method using SAO and deconvolving signals from nuclei, according to another embodiment. Some or all of the steps in FIG. 11 can be performed by a computing system. In the embodiment of FIG. 11, the imaging system and method performs additional operations to acquire signals from cell nuclei or autofluorescence from endogenous processes within the cell, and deconvolves these signals from signals acquired from the actual target to obtain an improved high-resolution target spatial map compared to when these signals are not decoupled.

In one primary example referred throughout the specification, the structured illumination patterns used to illuminate the target for each channel uses N=4 distinct 2D excitation patterns and M=3 phases, yielding 12 raw images per acquisition channel. In this context, each raw image comprises pixel-level signals indexed by i.

In one embodiment, a plurality of raw target images are acquired by measuring the optical signals from the target illuminated with a first plurality of structured illumination patterns using a first acquisition channel. Each raw target image includes at least one raw target intensity value obtained from measurements of the target responsive to being illuminated with a corresponding structured illumination pattern. Therefore, the plurality of raw target images may be a collection of 12 raw images. In one instance, the first acquisition channel is configured to optimize for signals from proteins (e.g., antigens) in the target. As an example, the excitation wavelength may be less than 550 nm. In FIG. 11, the plurality of raw target images are denoted by:
[symbol] : Protein channel 12 images.

A plurality of raw nuclei images are also acquired by measuring optical signals from the target illuminated with a second plurality of structured illumination patterns using a second acquisition channel. Each raw nuclei image includes at least one raw nuclei intensity value obtained from measurements of nuclei in the target responsive to being illuminated with a corresponding structured illumination pattern. Therefore, the plurality of raw nuclei images may be a collection of 12 raw images. In one instance, the second acquisition channel is different from the first acquisition channel, and is configured to optimize for signals from nuclei in the target. As an example, the excitation wavelength may be larger than 550 nm. In FIG. 11, the plurality of raw nuclei images are denoted by:
[symbol] : Nuclei channel 12 images.

A nuclei S image is obtained by processing the plurality of raw nuclei images and applying an SAO(S) operation 1108. In one embodiment, given a collection of raw images (e.g., raw target images, raw nuclei images, or raw background images), the SAO(S) operation produces a same-resolution image indexed by i. The intensity value for a given pixel i is the average or approximately the average over the raw intensity values for the pixel i across the raw images obtained by illuminating the target with N×M structured illumination patterns. This is a uniform illumination approximation. In another embodiment, the intensity value for a given pixel i is based on a statistic (e.g., median, average) of the raw intensity values for the pixel i across the raw images. For example, in FIG. 11, the computation is denoted by:

\ddot{N}_{SS}=SAO_{S}( [symbol] ).

A plurality of deconvolved images is obtained by computing a difference between each of the raw target images and the nuclei S image. In one instance, the difference is computed by performing a subtract operation 1110. In FIG. 11, the computation is denoted by:

[symbol]

= [symbol] −\ddot{N}_{SS}.

A reconstructed image is generated by processing the plurality of deconvolved images. The reconstructed image includes a set of reconstructed intensity values, and has a higher resolution than the plurality of deconvolved images. In one embodiment, the reconstruction is performed by applying a SAO(H) operation 1112. In one instance, the SAO(H) operation 1112 is the SAO imaging described in detail with respect to FIGS. 4-7. Each pixel in the reconstructed image may contain multiple sub-pixel components (k, i) as described with respect to FIGS. 4-7. In one instance, the SAO(H) operation may also follow the unfolding principle to remove aliasing and recover sub-pixel intensities s_{k,i} from the measured intensities m_{k,i}. During SAO(H) reconstructions, each pixel location i is divided virtually into multiple sub-pixels (k, i) to synthesize a higher resolution image, as described in detail above. The constructed sub-pixel intensities represent local responses within raw pixel i, yielding four-fold spatial resolution enhancement compared to the original grid for raw images. Thus, the reconstructed image may have a number of sub-pixels that are four times the number of pixels in a raw image. In FIG. 11, the computation is denoted by:

\ddot{P}′_{SH}=SAO_{H}( [symbol] ).

Moreover, a plurality of raw background images are acquired by measuring optical signals from the target illuminated with a third plurality of structured illumination patterns using a third acquisition channel. Each raw background image includes at least one raw background intensity value obtained from measurements of autofluorescence in the target. Therefore, the plurality of raw background images may be a collection of 12 raw images representing endogenous fluorescence signals, which may arise from metabolic or non-metabolic cellular processes. In one instance, the third acquisition channel is different from the first acquisition channel or the second acquisition channel, and is configured to optimize for signals from these autofluorescence signals. As an example, the excitation wavelength may be less than 550 nm. In FIG. 11, the plurality of raw background images are denoted by:
[symbol] : AF channel 12 images.

A background S image is obtained by processing the plurality of raw background images and applying an SAO(S) operation 1116. For example, the background S image may be approximately the average over the raw intensity values of the raw background images. In FIG. 11, the computation is denoted by:

Ä_{SS}=SAO_{S}( [symbol] ).

The background S image is upscaled by resizing the S image to a higher resolution by applying a resizing operation 1118. In one embodiment, the upscaled image is 4 times the resolution of the background S image, and therefore, has 4 times as many pixels than the background S image to match the resolution of the reconstructed image. In FIG. 11, the computation is denoted by:

Ä_{U4}=Resize₄(Ä_{SS}).

In addition, a target S image is obtained by processing the plurality of raw background images and applying an SAO(S) operation 1120. For example, the target S image may be approximately the average over the raw intensity values of the raw target images. In FIG. 11, the computation is denoted by:

\ddot{P}_{SS}=SAO_{S}( [symbol] ).

A second deconvolved image is obtained by computing a difference between the target S image and the nuclei S image. In one instance, the difference is computed by performing a subtract operation 1122. In FIG. 11, the computation is denoted by:

\ddot{P}′_{SS}=\ddot{P}_{SS}−\ddot{N}_{SS}.

The second devolved image is upscaled by resizing the image to a higher resolution by applying a resizing operation 1124. In one embodiment, the upscaled image is 4 times the resolution of the second deconvolved image, and therefore, has 4 times as many pixels than the second deconvolved image to match the resolution of the reconstructed image. In FIG. 11, the computation is denoted by:

\ddot{P}′_{U4}=Resize₄(\ddot{P}′_{SS}).

In one embodiment, a weight tensor is obtained that includes weight values indicating how the reconstructed intensity values in the reconstructed image should be weighted for the resulting target spatial map. Specifically, in one instance, the weight tensor is computed such that for a particular pixel location corresponding to a respective region of the target, the weight value for the pixel location is inversely proportional to an intensity value for the pixel location in the upscaled background S image. Since the intensity values in the upscaled background S image are indicative of autofluorescence signals in the respective region of the target, the weight value for a particular region decreases the signal for the region in the resulting target spatial map when the autofluorescence signal is high, and increases the signal for the region in the resulting target spatial map when the autofluorescence signal is low. This essentially suppresses noise. The number of weight values may correspond to the number of pixels (or sub-pixels) in the reconstructed image.

In one instance, the weight values are computed such that for a particular pixel location corresponding to a respective region of the target, the weight value is proportional to an intensity value for the pixel location in the upscaled second deconvolved image.

In one embodiment, the weight tensor is obtained by applying a ratio (i.e., division) operation 1126 of the intensity values in the upscaled second deconvolved image divided by the intensity values in the upscaled background S image on a per pixel basis. In FIG. 11, the computation is denoted by:

\ddot{W}_{U4}=\ddot{P}′_{U4}/Ä_{U4}.

Further, the target spatial map is obtained by for each particular pixel location, multiplying 1114 the weight value for the pixel with the reconstructed intensity value of the pixel in the reconstructed image. In FIG. 11, the computation is denoted by:

\ddot{P}ₘₐₚ=\ddot{W}_{U4}·\ddot{P}′_{SH}.

The target spatial map may be further rendered on a display of a client device or presented in some other medium for a user to view.

Conventional brightfield microscopy for chromogenic IHC imaging is often limited in spatial resolution and dynamic range. By applying structured excitation at multiple wavelengths to fluorescent imaging of chromogenic slides, the imaging system and methods described herein provides high-resolution spatial mapping at even single-cell level, enabling quantitative biomarker analysis. It has been demonstrated that computational pathology spatial image analysis of low-resolution brightfield IHC images has potential to improve patient selection and clinical outcomes for patients treated with TROP2 ADC datopotomab deruxtecan (Dato-Dx) (Garassino et al., WCLC 2024). The improved resolution and dynamic range of the embodiments disclosed herein can greatly enhance such approaches.

Applications of the disclosure include development of improved clinical biomarkers and diagnostics leveraging existing chromogenic reagents, re-analysis of archived samples for enhanced biomarker insights, generation of high-resolution training data for machine learning models in pathology, and correlation of protein localization with clinical, genomic, and transcriptomic data for target discovery.

### Examples of Target Spatial Map

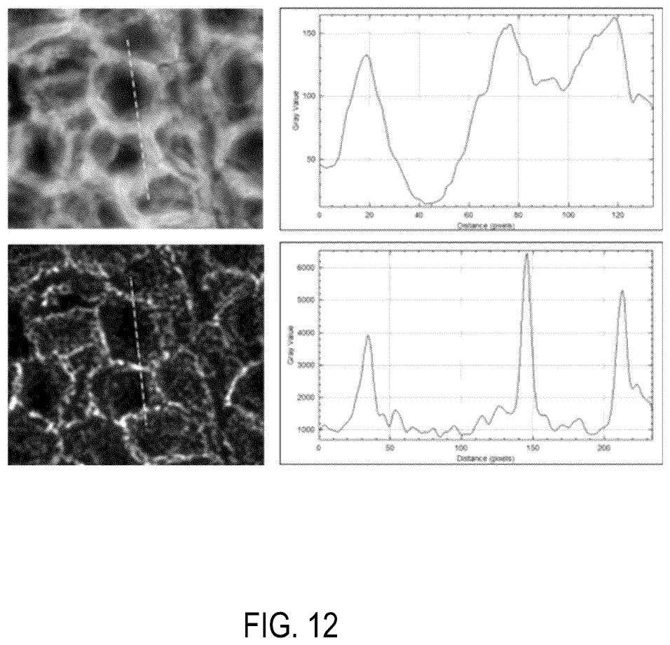

FIG. 12 illustrates examples of imaging methods that were performed human breast cancer tissue samples, according to one embodiment. Specifically, FIG. 12 illustrates an identical region of the human breast cancer tissue. The top row presents a spatial map imaged using conventional digital pathology techniques based on brightfield microscopy. The bottom row is imaged using the method described with respect to FIG. 11 herein. The method described herein provides higher spatial resolution and larger dynamic range for quantitative analysis at single cell level.

Upon reading this disclosure, those of skill in the art will appreciate still additional alternative structural and functional designs for a method and an apparatus for synthetic aperture optics. Thus, while particular embodiments and applications of the present invention have been illustrated and described, it is to be understood that the invention is not limited to the precise construction and components disclosed herein and that various modifications, changes and variations which will be apparent to those skilled in the art may be made in the arrangement, operation and details of the method and apparatus of the present invention disclosed herein without departing from the spirit and scope of the invention as defined in the appended claims.

What is claimed is:

1. A method for detecting protein on a target, the method comprising the steps of:
generating a plurality of raw target images by measuring optical signals from a target illuminated with a first plurality of structured illumination patterns, each raw target image including at least one raw target intensity value obtained from measurements of the target responsive to being illuminated with a corresponding structured illumination pattern;
generating a plurality of raw nuclei images by measuring optical signals from the target illuminated with a second plurality of structured illumination patterns, each raw nuclei image including at least one raw nuclei intensity value obtained from measurements of nuclei in the target responsive to being illuminated with a corresponding structured illumination pattern;
obtaining a nuclei S image by processing the plurality of nuclei raw images;
obtaining a plurality of deconvolved images by computing a difference between each of the raw target images and the nuclei S image;
generating a reconstructed image by processing the plurality of deconvolved images, wherein the reconstructed image includes a set of reconstructed intensity values, and wherein the reconstructed image has a higher resolution than the plurality of deconvolved images;
generating a target map from at least the reconstructed image of the target; and
displaying information obtained from the protein map on a client device.

2. The method of claim 1, wherein the target is a chromogenic immunohistochemistry (IHC) sample.

3. The method of claim 1, wherein the proteins in the target are stained with a first stain material, wherein the nuclei in the target are stained with a second stain material different from the first stain material.

4. The method of claim 3, wherein the first stain material is DAB (3,3′-Diaminobenzidine) and the second stain material is hematoxylin.

5. The method of claim 3, generating the protein map further comprising:
obtaining weight values indicating how the reconstructed intensity values in the reconstructed image should be weighted; and
for a particular pixel location, multiplying the weight value for the pixel location with the reconstructed intensity value for the pixel location in the reconstructed image to generate the target map.

6. The method of claim 5, further comprising:
generating a plurality of raw background images by measuring optical signals from the target illuminated with a third plurality of structured illumination patterns, each raw background image including at least one raw background intensity value obtained from measurements of autofluorescence in the target; and
obtaining an upscaled background S image by processing the plurality of raw background images,
wherein the weight value for the pixel location corresponding to a respective region of the target is inversely proportional to an intensity value for the pixel location in the upscaled background S image indicative of autofluorescence in the respective region of the target.

7. The method of claim 6, further comprising:
obtaining a protein S image by processing the plurality of raw protein images;
obtaining a second deconvolved image by computing a difference between the protein S image and the nuclei S image; and
obtaining an upscaled second deconvolved image,
wherein the weight value for the pixel location corresponding to the respective region of the target is proportional to an intensity value for the pixel location in the upscaled second deconvolved image.

8. The method of claim 1, wherein each of the first plurality, the second plurality, and the third plurality of structured illumination patterns are selective excitation patterns with a set of illumination characteristics including at least a spatial frequency and phase of the selective excitation pattern.

9. A system, comprising:
a plurality of illumination modules configured to illuminate the target with a first plurality of structured illumination patterns and a second plurality of structured illumination patterns;
an optical imaging module configured to:
generate a plurality of raw target images by measuring optical signals from the target illuminated with the first plurality of structured illumination patterns, each raw target image including at least one raw target intensity value obtained from measurements of the target responsive to being illuminated with a corresponding structured illumination pattern, and
generate a plurality of raw nuclei images by measuring optical signals from the target illuminated with the second plurality of structured illumination patterns, each raw nuclei image including at least one raw nuclei intensity value obtained from measurements of the target responsive to being illuminated with a corresponding structured illumination pattern;
an imaging module configured to:
obtain a nuclei S image by processing the plurality of nuclei raw images,
obtain a plurality of deconvolved images by computing a difference between each of the raw target images and the nuclei S image,
generate a reconstructed image by processing the plurality of deconvolved images, wherein the reconstructed image includes a set of reconstructed intensity values, and wherein the reconstructed image has a higher resolution than the plurality of deconvolved images,
generate a target map from at least the reconstructed image of the target and
display information obtained from the protein map on a client device.

10. The system of claim 9, wherein target is a chromogenic immunohistochemistry (IHC) sample.

11. The system of claim 9, wherein the proteins in the target are stained with a first stain material, wherein the nuclei in the target are stained with a second stain material different from the first stain material.

12. The system of claim 11, wherein the first stain material is DAB (3,3′-Diaminobenzidine) and the second stain material is hematoxylin.

13. The system of claim 9, wherein the imaging module is further configured to:
obtain weight values indicating how the reconstructed intensity values in the reconstructed image should be weighted; and
for a particular pixel location, multiply the weight value for the pixel location with the reconstructed intensity value for the pixel location in the reconstructed image to generate the target map.

14. The system of claim 13, wherein the imaging module is further configured to:
generate a plurality of raw background images by measuring optical signals from the target illuminated with a third plurality of structured illumination patterns, each raw background image including at least one raw background intensity value obtained from measurements of autofluorescence in the target; and
obtain an upscaled background S image by processing the plurality of raw background images,
wherein the weight value for the pixel location corresponding to a respective region of the target is inversely proportional to an intensity value for the pixel location in the upscaled background S image indicative of autofluorescence in the respective region of the target.

15. The system of claim 14, wherein the imaging module is further configured to:
obtain a protein S image by processing the plurality of raw protein images;
obtain a second deconvolved image by computing a difference between the protein S image and the nuclei S image; and
obtain an upscaled second deconvolved image,
wherein the weight value for the pixel location corresponding to the respective region of the target is proportional to an intensity value for the pixel location in the upscaled second deconvolved image.

16. The system of claim 9, wherein each of the first plurality, the second plurality, and the third plurality of structured illumination patterns are selective excitation patterns with a set of illumination characteristics including at least a spatial frequency and phase of the selective excitation pattern.
